(19) United States
(12) Patent Application Publication
(10) Pub. No.: US 2024/0269367 A1
(43) Pub. Date: Aug. 15, 2024

(54) PERFUSION STATE DETECTION METHOD, PERFUSION STATE DETECTION APPARATUS, AND ENDOSCOPE SYSTEM

(71) Applicant: OLYMPUS CORPORATION, Tokyo (JP)

(72) Inventor: Shoei TSURUTA, Tokyo (JP)

(73) Assignee: OLYMPUS CORPORATION, Tokyo (JP)

(21) Appl. No.: 18/438,757

(22) Filed: Feb. 12, 2024

Related U.S. Application Data
(63) Continuation of application No. PCT/JP2021/031424, filed on Aug. 26, 2021.

(51) Int. Cl. A61M 1/00; A61B 1/00; A61B 1/015; A61B 1/12

(52) U.S. Cl. CPC A61M 1/84; A61B 1/00068; A61B 1/015; A61B 1/126

(57) ABSTRACT

A perfusion state detection method includes: driving a liquid feeding pump configured to supply a liquid via a liquid feeding conduit inserted into a living body; driving a suction pump configured to discharge the liquid via a suction conduit inserted into the living body; obtaining a distance, on a plane indicating a relation between any two values of a drive output to the suction pump, a flow rate of the liquid flowing through the conduit, and a pressure in the conduit, between a coordinate on the plane of the two values in a perfusion state at normal time of the conduit and a coordinate on the plane of the two values actually measured; detecting abnormality of the perfusion state of the conduit based on whether the distance exceeds a threshold; and controlling a flow of the liquid in the conduit based on a detection result of the perfusion state.

### CROSS REFERENCE TO RELATED APPLICATION

This application is a continuation application of PCT/JP2021/031424 filed on Aug. 26, 2021, the entire contents of which are incorporated herein by this reference.

### BACKGROUND OF THE INVENTION

### 1. Field of the Invention

The present invention relates to a perfusion state detection method, a perfusion state detection apparatus, and an endoscope system that can be used as an apparatus that collects calculus in a subject.

### 2. Description of the Related Art

Conventionally, as an apparatus for collecting calculus from the inside of a body, a calculus collecting apparatus has been developed in which a laser light is used to grind the calculus and the crushed calculus pieces are collected. For example, a technique has been proposed in which a laser light is irradiated from a laser probe that is inserted through a treatment instrument channel of an endoscope to finely grind the calculus. In the proposal, the grinded calculus (crushed stone) is grasped by forceps to be extracted to the outside of a body.

Further, there is also a calculus treatment system that collects calculus by performing water feeding and suction. In this system, the calculus is perfused together with water via a suction tube to be collected to the outside of the body. However, calculus is occasionally caught in the suction tube. If so, starting from the caught calculus, the subsequent calculus is caught, which finally could lead to clogging of the suction tube.

Thus, Japanese Patent Application Laid-Open Publication No. 2018-166725 discloses a technique of assuming the presence of clogging of a suction line by monitoring a temporal change in the suction pressure of the suction line.

### SUMMARY OF THE INVENTION

A perfusion state detection method according to one aspect of the present invention includes: driving a liquid feeding pump configured to supply a liquid via a liquid feeding conduit inserted into an inside of a living body; driving a suction pump configured to discharge the liquid via a suction conduit inserted into the inside of the living body; obtaining a distance, on a plane indicating a relation between any two values of a drive output to the suction pump, a flow rate of the liquid flowing through the conduit, and a pressure in the conduit, between a coordinate on the plane of the two values in a perfusion state at normal time of the conduit and a coordinate on the plane of the two values actually measured; detecting abnormality of the perfusion state of the conduit based on whether the distance exceeds a threshold; and controlling a flow of the liquid in the conduit based on a detection result of the perfusion state.

A perfusion state detection apparatus according to one aspect of the present invention includes: a liquid feeding conduit inserted into an inside of a living body and configured to feed a liquid to the inside of the living body; a liquid feeding pump configured to supply the liquid to the liquid feeding conduit; a suction conduit inserted into the inside of the living body and configured to suck the liquid from the inside of the living body; a suction pump configured to discharge the liquid from the inside of the living body via the suction conduit; a valve configured to generate reverse jet in the suction conduit by a water hammering action; and a processor, in which the processor obtains a distance, on a plane indicating a relation between any two values of a drive output to the suction pump, a flow rate of the liquid flowing through the suction conduit, and a suction pressure in the suction conduit, between a coordinate on the plane of the two values in a perfusion state at normal time of the conduit and a coordinate on the plane of the two values actually measured; detects abnormality of the perfusion state of the suction conduit based on whether the distance exceeds a first threshold; and controls opening and closing of the valve based on a detection result of the perfusion state to generate the reverse jet in the suction conduit.

An endoscope system according to one aspect of the present invention includes an endoscope; a liquid feeding pump configured to cause a liquid to flow to a liquid feeding conduit of the endoscope; a suction pump configured to cause the liquid to flow via a suction conduit of the endoscope; a flow meter provided in the suction conduit and configured to measure a flow rate of the liquid flowing through the suction conduit; a valve configured to generate reverse jet in the suction conduit by a water hammering action; and a processor, in which the processor: obtains a distance, on a plane indicating a relation between any two values of a drive output to the suction pump, the flow rate of the liquid flowing through the suction conduit, and a suction pressure in the suction conduit, between a coordinate on the plane of the two values in a perfusion state at normal time of the conduit and a coordinate on the plane of the two values actually measured; detects abnormality of the perfusion state of the suction conduit based on whether the distance exceeds a first threshold; and controls opening and closing of the valve based on a detection result of the perfusion state to generate the reverse jet in the suction conduit.

### Advantageous Effect of Invention

According to the present invention, there is an advantageous effect of enabling to prevent a suction tube from being clogged.

### BRIEF DESCRIPTION OF THE DRAWINGS

FIG. 1 is a schematic configurational view showing a medical system including a medical apparatus according to a first embodiment of the present invention;

FIG. 2 is a block diagram showing a configuration of the medical apparatus including a perfusion state detection apparatus;

FIG. 3 is an explanatory view for explaining a distal end portion of an insertion portion of an endoscope;

FIG. 4 is an explanatory view for explaining the distal end portion of the insertion portion of the endoscope;

FIG. 5 is an explanatory view for explaining perfusion state detection by a perfusion state detection circuit 14;

FIG. 6 is a flowchart for explaining perfusion control by a medical apparatus 10;

FIG. 7 is an explanatory view for explaining operation of a modification;

FIG. 8 is a flowchart for explaining the operation of the modification;

FIG. 9 is a flowchart for explaining operation of another modification;

FIG. 10 is a block diagram showing a second embodiment of the present invention;

FIG. 11 is an explanatory view for explaining a technique of perfusion state detection by the perfusion state detection circuit 14 in the second embodiment;

FIG. 12 is a flowchart for explaining perfusion control by a medical apparatus 10A;

FIG. 13 is a block diagram showing another modification;

FIG. 14 is a block diagram showing another modification;

FIG. 15 is a flowchart for explaining operation of the modification of FIG. 14;

FIG. 16 is a block diagram showing a third embodiment of the present invention; and

FIG. 17 is an explanatory view for explaining a technique of perfusion state detection by the perfusion state detection circuit 14 in the third embodiment.

### DETAILED DESCRIPTION OF THE PREFERRED EMBODIMENTS

Hereinafter, embodiments of the present invention will be described in detail with reference to the drawings.

### First Embodiment

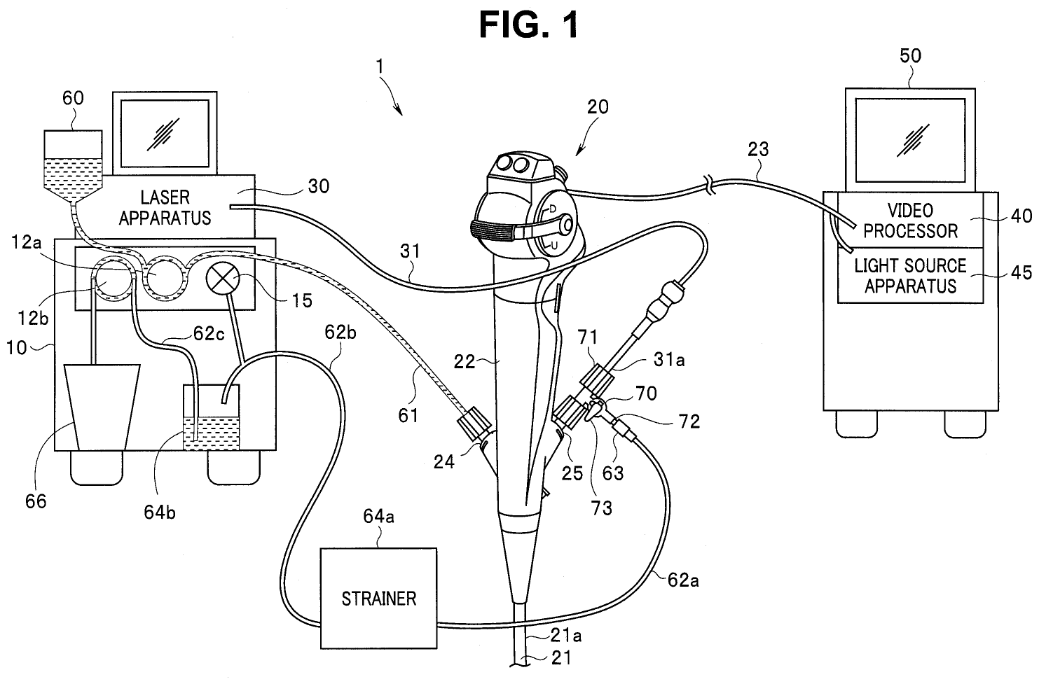

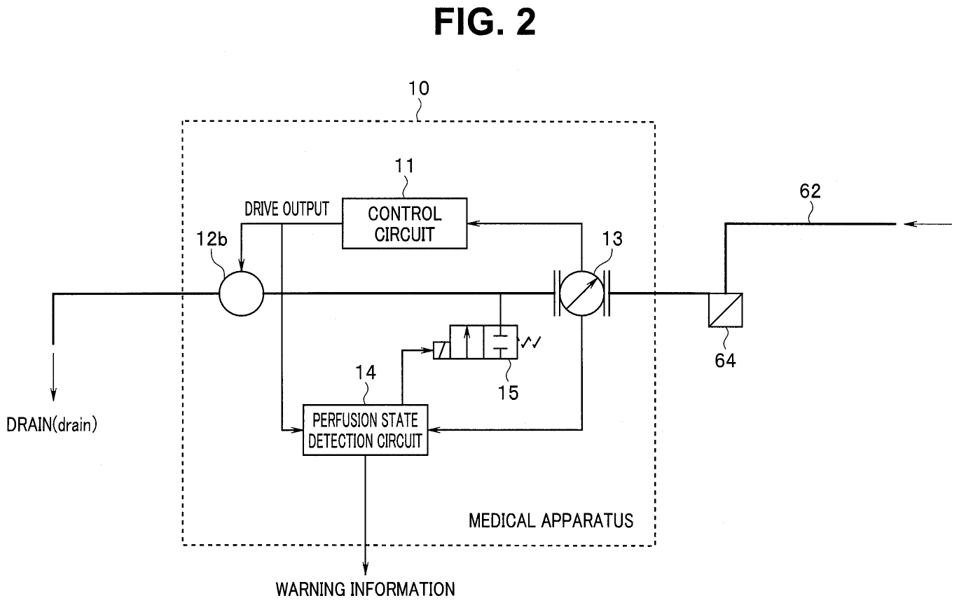

FIG. 1 is a schematic configurational view showing a medical system including a medical apparatus according to a first embodiment of the present invention. FIG. 2 is a block diagram showing a configuration of the medical apparatus including a perfusion state detection apparatus. The present embodiment detects the state of perfusion based on the relation between the flow rate in a suction conduit for discharging calculus to the outside of a body and the pump drive output, so that the calculus being caught can be detected at an early stage.

With reference to FIG. 1, a medical system 1 including a medical apparatus 10 will be described.

As shown in FIG. 1, the medical system 1 includes the medical apparatus 10, an endoscope 20, a laser apparatus 30, a video processor 40, a light source apparatus 45, and a monitor 50. The endoscope 20 includes an elongated insertion portion 21 and an operation portion 22. In the endoscope 20, the insertion portion 21 is inserted into an organ of a subject, for example, the kidney, and an image of the organ is picked up and an image pickup signal is outputted.

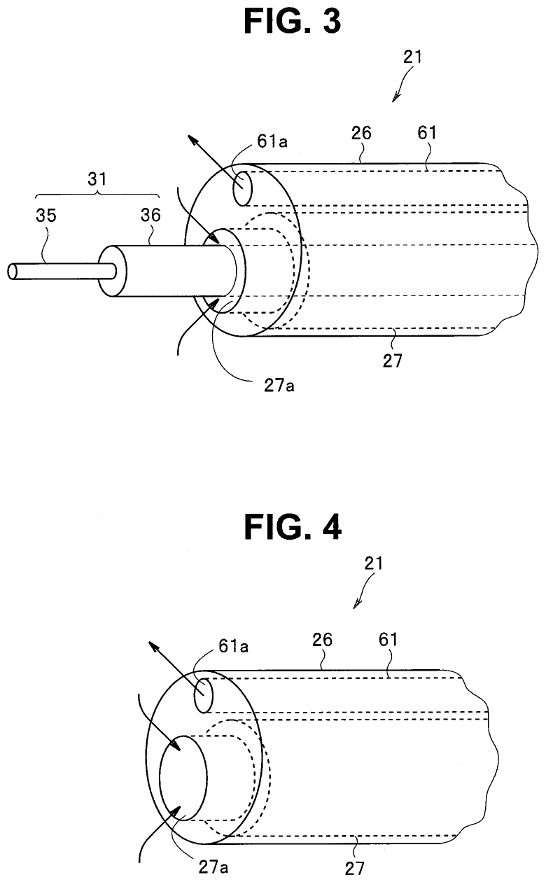

The insertion portion 21 is configured, for example, with a flexible portion 21a on a proximal end side, a bending portion, which is not shown, on a distal end side of the flexible portion 21a, and a rigid distal end portion 26 (see FIG. 3) on a distal end side of the bending portion that are continuously provided. On the proximal end side of the insertion portion 21, the operation portion 22 provided with various buttons for operating the endoscope 20 is disposed. Note that the bending portion is configured to bend with the operation of the operation portion 22.

One end of a universal cord 23 is connected to the operation portion 22 and the other end of the universal cord 23 is connected to the video processor 40 and the light source apparatus 45. The endoscope 20 is connected to the video processor 40 and the light source apparatus 45 by means of the universal cord 23, so that various signals and illumination light are transmitted.

The video processor 40 controls the entire medical system 1. An image pickup signal is inputted to the video processor 40 from the endoscope 20 via the universal cord 23 and the video processor 40 obtains an image signal by performing signal processing on the inputted image pickup signal. The video processor 40 outputs the image signal to the monitor 50. The monitor 50 displays an image based on the image signal outputted by the processor 40.

The light source apparatus 45 includes, for example, a white LED, and emits illumination light. The illumination light emitted by the light source apparatus 45 is guided to the rigid distal end portion 26 via the universal cord 23 and a light guide (not shown) inserted through the insertion portion 21.

The operation portion 22 is provided with a liquid feeding tube attaching pipe sleeve 24 and a T-shaped tube attaching pipe sleeve 25. A liquid feeding tube 61 connected to a tank 60 is connected to the liquid feeding tube attaching pipe sleeve 24. The liquid feeding tube 61 is inserted through the inside of the insertion portion 21 up to the distal end of the rigid distal end portion 26.

Further, the operation portion 22 includes an opening portion communicating with a suction channel 27 (see FIG. 3) provided inside the insertion portion 21 and the opening portion is provided with the T-shaped tube attaching pipe sleeve 25. A T-shaped tube 70 is attached to the T-shaped tube attaching pipe sleeve 25. The T-shaped tube 70 is provided with a fiber for laser attaching port 71. A fiber attaching portion 31a for a fiber for laser 31 connected to the laser apparatus 30 is attached to the fiber for laser attaching port 71. The fiber for laser 31 can be inserted through the inside of the suction channel 27 via the T-shaped tube 70 and the T-shaped tube attaching pipe sleeve 25.

The T-shaped tube 70 is provided with a drain pipe sleeve 72. A tube attaching portion 63 for a suction tube 62a is attached to the drain pipe sleeve 72. The T-shaped tube 70 is provided with a cock 73, and the cock 73 causes water sacked from the suction channel 27 to flow toward the suction tube 62a side so as to prevent the water from flowing toward the fiber for laser attaching port 71 side.

The suction tube 62a is connected to a secondary strainer 64b via a primary strainer 64a and a suction tube 62b. The secondary strainer 64b is connected to a drain tank 66 via a suction tube 62c. Note that the suction tubes 62a, 62b, 62c are occasionally referred to as a suction tube 62 without being distinguished from one another. The suction tube 62a, the suction tube 62b, and the suction tube 62c may be coupled together without the primary strainer 64a and the secondary strainer 64b.

The medical apparatus 10 is provided with a liquid feeding pump 12a and a suction pump 12b. The liquid feeding pump 12a and the suction pump 12b may be configured with, for example, a tube pump. The liquid feeding pump 12a supplies liquid filled in the tank 60 to an organ in a body via the liquid feeding tube 61. The suction pump 12b is connected to the suction tube 62a via the suction tube 62c, the secondary strainer 64b, the suction tube 62b, and the primary strainer 64a, and the negative pressure of the suction tube 62c by means of the suction pump 12b is transmitted to the suction tube 62a. In other words, the liquid sucked from the organ in the body is discharged, by means of the suction pump 12b, to the drain tank 66 via the suction channel 27, the suction tube 62a, the primary strainer 64a, the suction tube 62b, the secondary strainer 64b, and the suction tube 62c. Note that the primary strainer 64a and the secondary strainer 64b are occasionally referred to as a strainer 64 without being distinguished from each other.

FIG. 3 and FIG. 4 are explanatory views for explaining a distal end portion of an insertion portion of an endoscope.

In the rigid distal end portion 26 of the insertion portion 21, on the distal end surface, an illumination window (not shown) where a distal end surface of the light guide faces and an observation window (not shown) to guide an optical image of a subject to a light receiving surface of an image pickup device, which is not shown, are disposed. In the present embodiment, on the distal end surface of the rigid distal end portion 26, a distal end opening portion 61a of the liquid feeding tube 61 is disposed. Arrows shown in the distal end opening portion 61a of FIG. 3 and FIG. 4 indicate that the liquid is ejected through the distal end opening portion 61a of the liquid feeding tube 61. The liquid (physiological salt solution) stored in the tank 60 is fed, by means of the liquid feeding pump 12a, to the organ in the body from the distal end surface of the rigid distal end portion 26 via the liquid feeding tube 61 inserted through the inside of the insertion portion 21.

Further, on the distal end surface of the rigid distal end portion 26, a distal end opening portion 27a of the suction channel 27 is disposed. Arrows shown in the distal end opening portion 27a of FIG. 3 and FIG. 4 indicate that the liquid in the organ in the body is sucked by the suction channel 27. The liquid in the organ in the body is discharged, by means of the suction pump 12b, to the drain tank 66 via the suction channel 27, the suction tube 62a, the primary strainer 64a, the suction tube 62b, the secondary strainer 64b, and the suction tube 62c.

Note that in the present embodiment, the example of using the suction channel 27 and the suction tube 62 as a suction conduit is shown, but the drainage from the organ to the outside may be performed such that a suction tube is inserted through the inside of the suction channel 27 and is extended to the outside via the T-shaped tube 70 so as to be used as the suction conduit.

In the example of FIG. 3, the fiber for laser 31 inserted from the T-shaped tube 70 is inserted through the inside of the suction channel 27 and is disposed inside the suction channel 27, with the distal end projecting from the distal end surface of the rigid distal end portion 26. Note that the fiber for laser 31 is configured with a core-clad 35 and a jacket 36 covering the core-clad 35. The laser apparatus 30 irradiates a laser light from a distal end of the fiber for laser 31 via the fiber for laser 31.

As shown in FIG. 3, at the time of collecting calculus, the fiber for laser 31 is inserted through the inside of the suction channel 27 and with the distal end of the fiber for laser 31 projecting from the distal end opening portion 27a, an endoscope image of the inside of the organ is obtained by the endoscope 20. In other words, the illumination light guided by the light guide, which is not shown, is illuminated to a subject through the illumination window, which is not shown, on the distal end surface of the rigid distal end portion 26. The reflected light of the subject passes through the observation window, which is not shown, and is received by the image pickup device. The image pickup device acquires an image pickup signal based on an optical image of the subject and outputs the image pickup signal to the video processor 40 via a cable, which is not shown, inside the insertion portion 21 and the universal cord 23. The video processor 40 displays the endoscope image based on the image pickup signal on the monitor 50. In this manner, an operator can observe, on the monitor 50, the inside of the organ where the rigid distal end portion 26 is disposed. The operator irradiates the calculus with a laser by directing the distal end of the fiber for laser 31 toward the calculus inside the organ and operating the laser apparatus 30 while viewing the endoscope image. With the laser irradiated, the calculus is grinded into relatively small grinded pieces.

In the present embodiment, in the state shown in FIG. 3, the liquid is discharged from the inside of the organ while feeding water to the inside of the organ, with the action of the liquid feeding pump 12a and the suction pump 12b. With such perfusion activity, the calculus inside the organ is sucked into the suction channel 27 through a gap between the fiber for laser 31 inserted through the inside of the suction channel 27 and an inner surface of the suction channel 27 and is discharged to the suction tube 62a via the T-shaped tube 70.

When the laser irradiation by the fiber for laser 31 ends, the fiber for laser 31 is withdrawn through the fiber for laser attaching port 71. In this manner, as shown in FIG. 4, the fiber for laser 31 is removed from the suction channel 27. Thereafter, the calculus is discharged to the outside of the body via the suction channel 27 that is relatively wide.

Since the calculus is collected with the fiber for laser 31 being inserted through the inside of the suction channel 27, the calculus passes through a relatively narrow drain path between the fiber for laser 31 and the inner surface of the suction channel 27 and the calculus is likely to be caught between the suction channel 27 and the fiber for laser 31. Note that the suction channel 27 is a relatively narrow drain path, and even when the suction is performed with the fiber for laser 31 being removed from the suction channel 27 as in FIG. 4, the calculus is occasionally caught in the suction channel 27. Once the calculus is caught, starting from the caught calculus, the subsequent calculus is caught, and the suction channel 27 is likely to be finally clogged. For example, if such clogging of the suction channel 27 occurs while collecting the calculus inside the kidney, increase in the pressure inside the renal pelvis is concerned in some cases.

Thus, in the present embodiment, the state of perfusion is monitored so as to enable to detect the calculus being caught at an early stage.

### (Configuration of Medical Apparatus 10)

In FIG. 2, the medical apparatus 10 includes a control circuit 11, the suction pump 12b, a flow meter 13, a perfusion state detection circuit 14, and a solenoid valve 15. The perfusion state detection apparatus is configured with the control circuit 11, the flow meter 13, and the perfusion state detection circuit 14. In FIG. 2, the primary strainer 64a and the secondary strainer 64b of FIG. 1 are shown as the strainer 64, and the suction tubes 62a, 62b, 62c are shown as the suction tube 62.

The control circuit 11 and the perfusion state detection circuit 14 may be configured with a processor using a CPU (Central Processing Unit), a FPGA (Field Programmable Gate Array), and the like. The control circuit 11 and the perfusion state detection circuit 14 may operate in accordance with a program stored in a memory which is not shown or may implement part of or the entire function by an electronic circuit of hardware. Note that the control circuit 11 and the perfusion state detection circuit 14 may be configured with one processor or may be configured with a plurality of processors. The function of the perfusion state detection circuit 14 may be implemented by the control circuit 11.

The control circuit 11 controls each portion of the medical apparatus 10. The control circuit 11 generates drive output to drive the suction pump 12b to be outputted to the suction pump 12b. The suction pump 12b operates based on the drive output, thereby generating a predetermined suction pressure inside the suction conduit with the suction channel 27 and the suction tube 62. For example, assuming that a conduit resistance of the suction conduit with the suction channel 27 and the suction tube 62 (hereinafter, the suction channel 27 and the suction tube 62 are simply referred to as the suction conduit) is constant, the suction pump 12b can cause a liquid at a flow rate substantially proportional to the drive output to flow to the suction conduit. In other words, in this case, the flow rate in the suction conduit increases or decreases proportionally to the drive output.

The flow meter 13 is provided between the strainer 64 and the suction pump 12b in the middle of the suction conduit as the suction tube 62. The flow meter 13 measures the flow rate of the liquid flowing through a suction flow path with the suction tube 62 and outputs the measurement result to the control circuit 11 and the perfusion state detection circuit 14. Note that the flow rate in the suction conduit can be set by a user such as an operator using an input apparatus which is not shown. Alternatively, the control circuit 11 may be configured to set the flow rate in the suction conduit to a predetermined flow rate.

In the present embodiment, the control circuit 11 performs feedback control, such as PID (proportional-integral-derivative) control, to change the drive output to the suction pump 12b based on the measurement result from the flow meter 13 for maintaining the set flow rate. With the feedback control, even if the conduit resistance of the suction conduit fluctuates to some extent, the flow rate in the suction conduit can be maintained at the flow rate set by the user. As a result, the perfusion can be performed with a constant pressure inside the organ.

However, also in a case where the conduit resistance increases due to the calculus being caught in the suction conduit, such as the suction channel 27, the drive output to the suction pump 12b also increases against the decrease in the flow rate due to the increase in the conduit resistance, and the flow rate is maintained at the set flow rate. Meanwhile, when more calculi are caught and the conduit resistance excessively increases, the drive output to the pump 12b reaches the upper limit, so that the flow rate becomes lower than the set flow rate, which could finally result in the clogging state with a flow rate of 0.

Thus, in the present embodiment, the perfusion state detection circuit 14 detects the calculus being caught from the state of perfusion at an early stage. In other words, the perfusion state detection circuit 14 is provided with not only the measurement result of the flow rate from the flow meter 13, but also the drive output or information on the drive output (hereinafter simply referred to as the drive output) from the control circuit 11. The perfusion state detection circuit 14 is controlled by the control circuit 11 to detect the state of perfusion (hereinafter, referred to as the perfusion state) based on the liquid feeding to the inside of the organ via the liquid feeding tube 61 and the drainage via the suction conduit with the suction channel 27 and the suction tube 62, in accordance with the drive output from the control circuit 11 and the measurement result of the flow rate from the flow meter 13. The perfusion state detection circuit 14 determines, based on the detection result, for example, whether any abnormality has occurred in the perfusion state, such as whether the calculus is caught in the suction conduit.

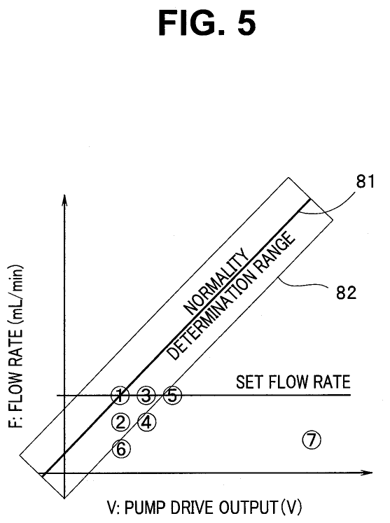

FIG. 5 is an explanatory view for explaining perfusion state detection by the perfusion state detection circuit 14. FIG. 5 shows the relation between a drive output V and a flow rate F on a V-F plane with the drive output V (V) to the suction pump 12b plotted on the lateral axis and with the flow rate F (mL/min) of the liquid flowing through the suction conduit plotted on the longitudinal axis. As described above, when the conduit resistance of the suction conduit is constant, the flow rate F also changes proportionally to the increase and decrease in the drive output to the suction pump 12b. A straight line 81 of FIG. 5 shows a V-F characteristic curve in a case of the conduit resistance of the suction conduit at normal perfusion state (hereinafter, referred to as an initial conduit resistance).

In other words, when the V-F characteristic of the drive output V obtained by the output from the control circuit 11 and the flow rate F obtained by the output from the flow meter 13 corresponds to the characteristic of the straight line 81, the conduit resistance of the suction conduit is assumed to be the initial conduit resistance at normal state. In other words, in this case, it is assumed that the conduit resistance is not increased due to calculus or the like being caught inside the suction conduit. For such detection of the perfusion state, the perfusion state detection circuit 14 obtains the relation between the drive output V obtained by the output from the control circuit 11 and the flow rate F obtained by the output from the flow meter 13.

In the present embodiment, the perfusion state detection circuit 14 determines that change in the conduit resistance is in the normal range for a range within a predetermined distance (hereinafter, referred to as a first determination threshold) from the straight line 81 indicating that the conduit resistance of the suction conduit is at normal state, that is, a normality determination range 82 of FIG. 5. Note that the normality determination range 82 takes into consideration the fluctuation of the conduit resistance due to normal bending of the insertion portion 21 or the like. The range of the normality determination range 82, that is, the degree of the first determination threshold can be appropriately set and changed. With the first determination threshold appropriately set, it is possible to adjust the degree of catching to be determined to be abnormal.

The perfusion state detection circuit 14 determines whether the obtained characteristic value of the drive output V-flow rate F is included in the normality determination range 82. Note that the conduit resistance of the suction conduit increases due to not only the calculus being caught, but also buckling of the suction conduit, absorption of a foreign object to the distal end opening portion, and the like. The perfusion state detection circuit 14 can also detect abnormality of the perfusion state in such cases.

The perfusion state detection circuit 14 may be configured to read, from a memory which is not shown, the first determination threshold for determining whether the characteristic value is included in the normality determination range 82. The user such as the operator may be able to set and change the first determination threshold by means of an input apparatus which is not shown.

When abnormality of perfusion is determined solely using the measurement result of the flow rate, detection of abnormality of perfusion may be impossible until the clogging of the suction conduit develops to become a substantially complete clogging by the feedback control of the suction pump 12b. By contrast, in the present embodiment, abnormality of perfusion is determined by combining the drive output V and the flow rate F, and abnormality of perfusion is determined without being interfered by a complicated flow and abnormality of perfusion that is merely only a partial clogging of the suction conduit by the calculus or the like can also be detected.

The suction tube 62 includes a bypass portion that blanches in the middle of a flow path between the flow meter 13 and the suction pump 12b, and the solenoid valve 15 is connected to a terminal end of the bypass portion. The solenoid valve 15 opens the suction tube 62 to the atmosphere in a fully-opened state, and closes the bypass portion in a fully-closed state. The perfusion state detection circuit 14 controls the opening and closing of the solenoid valve 15 based on the determination result of the abnormality in the conduit resistance of whether the characteristic value of the drive output V-flow rate F is included in the normality determination range 82. In other words, the perfusion state detection circuit 14 brings the solenoid valve into a fully-closed state when it is determined that no abnormality occurs in the perfusion (conduit resistance), and momentarily brings the solenoid valve 15 into a fully-opened state and then returns the solenoid valve 15 to the fully-closed state when it is determined that abnormality has occurred in the perfusion (conduit resistance).

The solenoid valve 15 is momentarily brought into a fully-opened state and then returned to a fully-closed state, so that a water hammering phenomenon is generated. Note that as a valve causing such a water hammering phenomenon, various types of valves can be adopted without being limited to the solenoid valve 15. With the water hammering phenomenon caused by the opening and closing of the solenoid valve 15, reverse jet to a fluid inside the suction conduit is generated. As a result, the calculus caught in the suction conduit is removed from the suction conduit with the liquid pressure by the reverse jet, so that the calculus being caught in the suction conduit is eliminated.

Note that when it is determined that abnormality has occurred in the conduit resistance, the perfusion state detection circuit 14 may be configured to output warning information indicating a possibility of clogging the suction conduit. For example, the monitor 50 may be configured to present warning display based on the warning information.

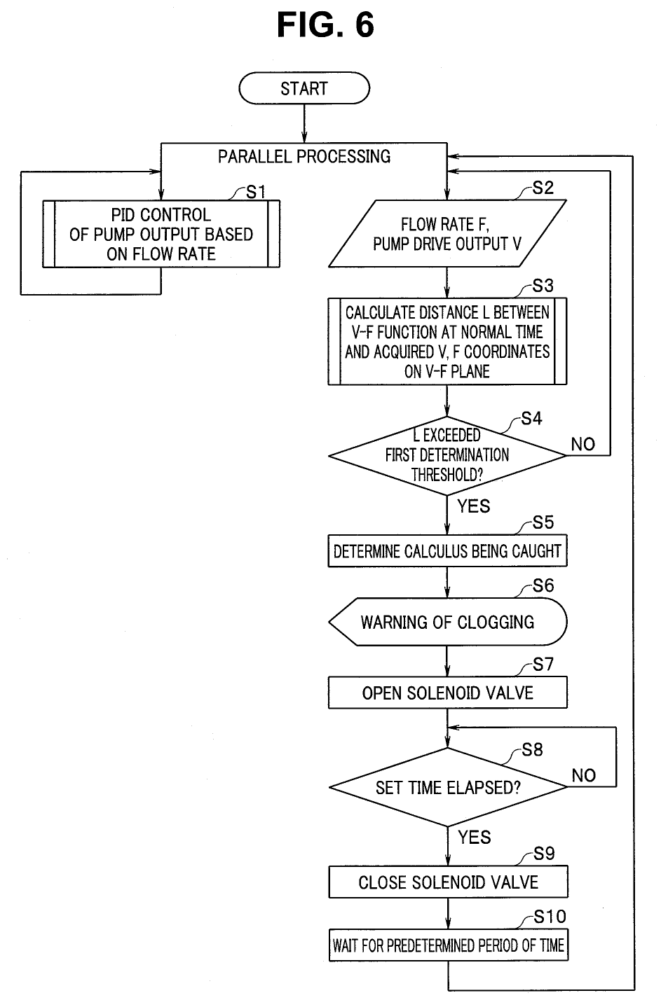

Next, the operation of the embodiment configured as such will be described with reference to FIG. 5 and FIG. 6. FIG. 6 is a flowchart for explaining perfusion control by the medical apparatus 10.

In step S1 of FIG. 6, the control circuit 11 performs PID control of the drive output V to the suction pump 12b so as to maintain the flow rate F at a set flow rate. Note that the control circuit 11 also performs the PID control of the liquid feeding pump 12a to maintain the set flow rate. In the present embodiment, the control circuit 11 performs processing of step S2 and the following processing in parallel to the control of step S1. Note that the processing of step S2 and the following processing are performed by the perfusion state detection circuit 14 that is controlled by the control circuit 11. The flow meter 13 measures the flow rate F of the liquid flowing through the suction conduit and outputs the measurement result to the control circuit 11 and the perfusion state detection circuit 14. The control circuit 11 performs the PID control of the drive output V based on the measurement result of the flow rate F (S1). The control circuit 11 provides the drive output V to be set for the suction pump 12b to the perfusion state detection circuit 14. The flow rate F and the drive output V are inputted to the perfusion state detection circuit 14 (S2).

The perfusion state detection circuit 14 calculates, in step S3, a distance L between a V-F function at normal time on a V-F plane indicated by the straight line 81 of FIG. 5 and an acquired coordinate value of the drive output V and the flow rate F. The perfusion state detection circuit 14 determines whether the distance L has exceeded the first determination threshold (step S4).

Now, for example, the coordinate value on the V-F plane of the drive output V and the flow rate F acquired by the perfusion state detection circuit 14 is denoted by a circled FIG. 1 of FIG. 5. Note that the set flow rate is the set flow rate shown in FIG. 5. The control circuit 11 performs feedback control of the drive output V based on the measurement result from the flow meter 13 for maintaining the set flow rate (S1). With this control, when the conduit resistance of the suction conduit does not change, the drive output V-flow rate F takes the coordinate value on the straight line 81 of FIG. 5 in accordance with the change in the set flow rate.

Here, for example, because of the normal bending operation of the insertion portion 21 or the like, the conduit resistance of the suction conduit increases from the initial conduit resistance. Thus, as shown in a circled FIG. 2 of FIG. 5, when the drive output V does not change, the flow rate F decreases. However, with the feedback control by the control circuit 11, the drive output V increases and irrespective of the change in the conduit resistance, the flow rate F returns to the set flow rate as shown in a circled FIG. 3 of FIG. 5. When the conduit resistance increases due to the normal bending operation of the insertion portion 21 or the like, the conduit resistance occasionally returns to the original initial conduit resistance, and in this case, with the feedback control by the control circuit 11, the drive output V and the flow rate F return to the coordinate value of the circled FIG. 1.

However, when the increase in the initial conduit resistance results from the calculus being caught inside the suction conduit or the like, subsequently, starting from the calculus being first caught, more calculi are caught and whereby the conduit resistance further increases in some cases. If so, the flow rate F decreases as shown in a circled FIG. 4 of FIG. 5, and with the feedback control by the control circuit 11, the drive output V increases (circled FIG. 5) and the set flow rate is maintained.

In the present embodiment, when the relation of the drive output V-the flow rate F deviates from the normality determination range 82, that is, when the distance L between the coordinate of the drive output V-flow rate F and the straight line 81 has exceeded the first determination threshold, the perfusion state detection circuit 14 determines that abnormality has occurred in the perfusion, that is, calculus has been caught (YES determination of S4). Note that when it is determined that the distance L is within the first determination threshold (NO determination of S4), the perfusion state detection circuit 14 returns the processing from step S4 to step S2.

In other words, in the example of FIG. 5, when the coordinate value of the drive output V-flow rate F is on the circled FIGS. 4, 5, the perfusion state detection circuit 14 determines in step S5 that calculus has been caught in the suction conduit. Note that when the flow rate does not return to the set flow rate even with the PID control, when more calculi are caught in a short period of time, and the like, the drive output V-flow rate F is occasionally shifted to the coordinate position of a circled FIG. 6 from the circled FIG. 1 of FIG. 5. Even in such cases, the perfusion state detection circuit 14 can determine that calculus has been caught, in a short period of time after the occurrence of such a failure.

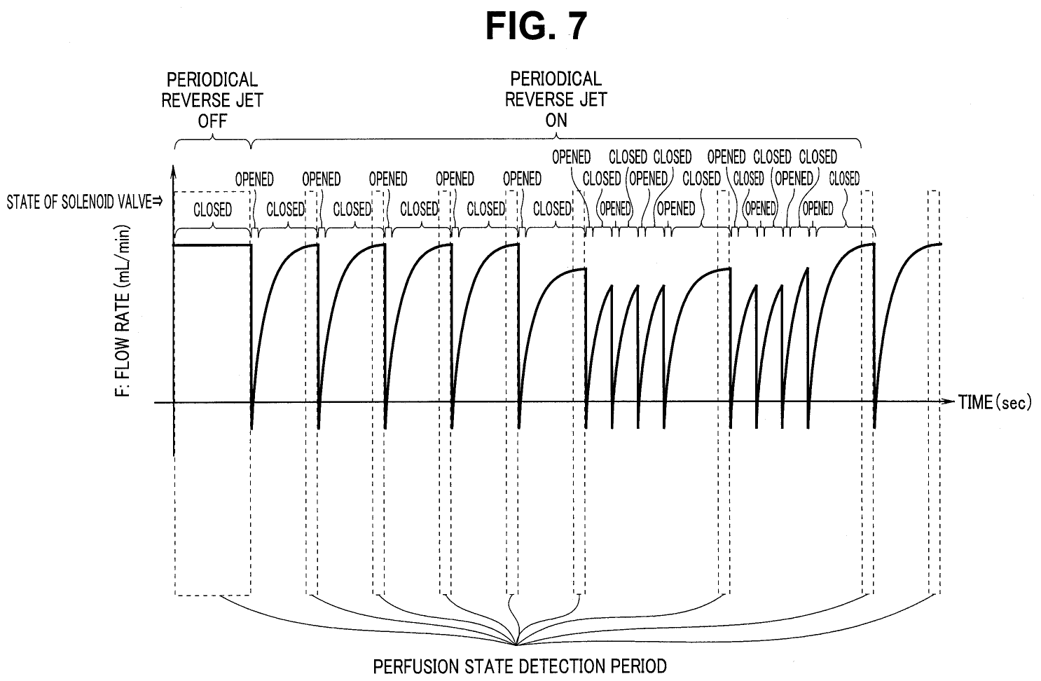

Note that it is also conceivable that despite the drive output V having reached the maximum value with the PID control by the control circuit 11, the flow rate does not return to the set flow rate and the drive output V-flow rate F is at the coordinate position of a circled FIG. 7 of FIG. 5. In this case also, the perfusion state detection circuit 14 can determine that calculus has been caught, before the suction conduit is completely clogged.

In the next step S6, the perfusion state detection circuit 14 outputs warning information indicating a possibility of clogging. In the next step S7, the perfusion state detection circuit 14 brings the solenoid valve 15 into a fully-opened state to open the suction conduit to the atmosphere, and after waiting for a set period of time in step S8, returns the solenoid valve 15 to a fully-closed state in step S9. Thus, the water hammering phenomenon occurs to generate the reverse jet in the suction conduit. In this manner, the calculus caught in the suction conduit is removed, so that the calculus being caught in the suction conduit is eliminated.

In the next step S10, the perfusion state detection circuit 14 waits for a predetermined period of time until the effect of the reverse jet on the conduit resistance disappears, and then returns the processing to step S2 and continues detection of the perfusion state. Thereafter, the same operation is repeated.

In this manner, in the present embodiment, the perfusion state is detected based on the relation between the flow rate in the suction conduit for discharging calculus to the outside of a body and the pump drive output, so that the calculus being caught can be detected at an early stage. In other words, minor catching of the calculus before leading to the clogging of the suction conduit can be detected at an early stage, so that a measure to resolve the state can be taken. For example, in the present embodiment, when abnormality of the perfusion state is detected, the suction conduit is opened to the atmosphere so as to generate the reverse jet by the water hammering action to be able to remove the calculus caught in the suction conduit. In this manner, it is possible to surely prevent the suction conduit from being clogged.

Note that in the first embodiment, the example of detecting the perfusion state based on the relation between the drive output V and the flow rate F has been shown, but since the suction pump is subjected to the PID control, when the set value of the flow rate is constant, it is also possible to detect abnormality of the perfusion state by simply comparing the drive output V of the pump with a predetermined threshold and based on whether the drive output V has exceeded the predetermined threshold.

Further, in FIG. 2, the solenoid valve 15 that opens the suction conduit to the atmosphere is brought into an open state and then into a closed state to generate the reverse jet, but the configuration can also be made such that the valve in an open state at normal state is provided in the middle of the flow path of the suction conduit, and the valve is brought into a closed state and is then returned to an open state to generate the reverse jet.

### (Modification)

FIG. 7 is an explanatory view for explaining operation of a modification. The hardware configuration of the present modification is the same as the hardware configuration of the first embodiment and the technique of detecting abnormality of the perfusion state is also the same as the technique of the first embodiment. In the present modification, to prevent the suction conduit from being clogged, the suction conduit is opened to the atmosphere at a predetermined interval to generate the reverse jet by the water hammering action. FIG. 7 shows the control of the present modification with time plotted on the lateral axis and with the flow rate F plotted on the longitudinal axis.

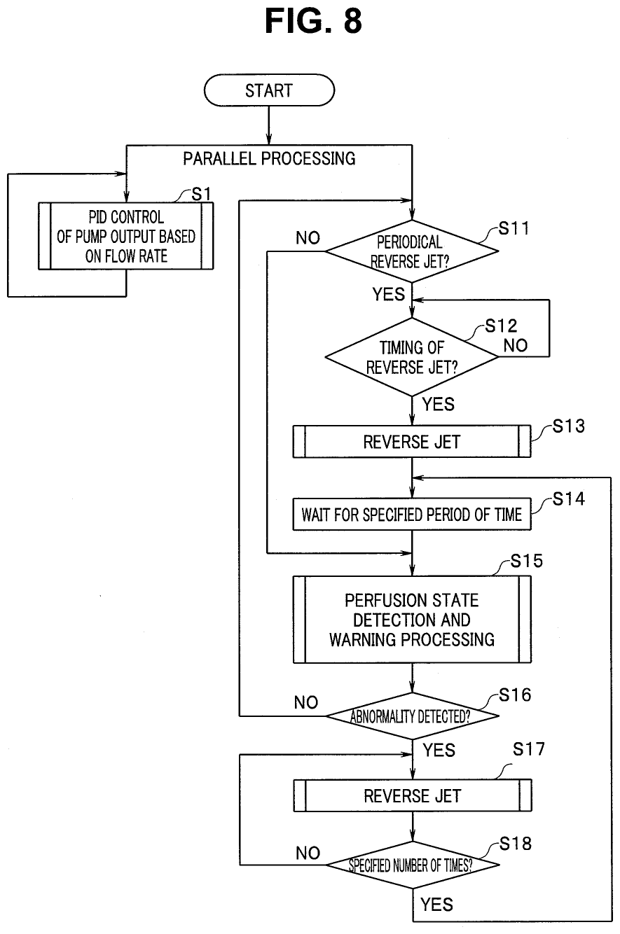

FIG. 8 is a flowchart for explaining the operation of the modification. In FIG. 8, for the same procedures as the procedures of FIG. 6, the same reference signs are assigned and the description will be omitted.

In step S11 of FIG. 8, the perfusion state detection circuit 14 determines whether periodical reverse jet is set. For example, the control circuit 11 can set a mode of performing periodical reverse jet (periodical reverse jet is ON) and a mode of not performing periodical reverse jet (periodical reverse jet is OFF) in accordance with an operator's operation or a predetermined sequence. Now, it is assumed that the periodical reverse jet is set to be OFF. The perfusion state detection circuit 14 shifts the processing to step S15 according to NO determination in step S11 and performs the perfusion state detection and warning processing. The processing of step S15 is the same as the processing of steps S2 to S6 of FIG. 6. In other words, the perfusion state detection circuit 14 determines whether the detection results of the drive output V and the flow rate F are deviated from the normality determination range 82, that is, whether the distance L between the V-F characteristic curve (straight line 81) at normal time on the V-F plane and the measured coordinate of the drive output V-flow rate F has exceeded the first determination threshold, so as to detect abnormality of the perfusion state.

In the next step S16, the perfusion state detection circuit 14 determines whether abnormality of the perfusion state has been detected. When abnormality of the perfusion state is not detected (NO determination of step S16), the perfusion state detection circuit 14 returns the processing to step S11. As shown in the initial perfusion state detection period of FIG. 7, in the state where no calculus is caught in the suction conduit, steps S11, S15, and S16 are repeated. In this case, with the PID control by the control circuit 11, the flow rate F is maintained at the set flow rate as shown in FIG. 7.

Next, as shown in FIG. 7, it is assumed that the periodical reverse jet is set to be ON. Thus, according to YES determination of step S11, the perfusion state detection circuit 14 shifts the processing to step S12 to determine whether the reverse jet timing has arrived. For example, the periodical reverse jet is performed at a predetermined cycle, and the perfusion state detection circuit 14 recognizes the reverse jet timing depending on whether the predetermined cycle has arrived. As shown in FIG. 7, when the periodical reverse jet is set to be ON, the first reverse jet is performed immediately afterwards.

In other words, the perfusion state detection circuit 14 shifts the processing to step S13 according to YES determination of step S12 and performs the reverse jet. The reverse jet of step S13 is the same processing as the processing of steps S7 to S9 of FIG. 6. Note that FIG. 7 shows that with the solenoid valve to be in an open state only for a short period of time, the suction conduit is opened to the atmosphere and the reverse jet is performed. As shown in FIG. 7, by performing the reverse jet, the flow rate F momentarily turns into negative. The negative flow rate F means that the liquid flows through the suction conduit in a direction reverse to the normal direction. This occasionally eliminates the calculus being caught.

In the next step S14, the perfusion state detection circuit 14 waits for a specified period of time and then in step S15, performs the perfusion state detection. As shown in FIG. 7, since the flow rate is significantly apart from the set flow rate due to the reverse jet, the perfusion state cannot be accurately detected even with the use of the drive output V and the flow rate F for a period until the effect of the reverse jet disappears. Thus, the perfusion state detection circuit 14 performs the perfusion state detection after the effect of the reverse jet is sufficiently reduced and the flow rate returns to the set flow rate. When abnormality of the perfusion state is not detected, the processing is returned from step S16 to step S11 and the same processing is repeated.

Here, it is assumed that calculus or the like is caught in the suction conduit. Thus, as a result of the perfusion state detection of step S15, abnormality of the perfusion state is detected. In this case, the perfusion state detection circuit 14 shifts the processing from step S16 to step S17 and performs the reverse jet. The reverse jet of step S17 is also the same processing as the processing of steps S7 to S9 of FIG. 6.

In the next step S18, the perfusion state detection circuit 14 determines whether the reverse jet has been performed a specified number of times. When the specified number of times of the reverse jet has not been reached, the perfusion state detection circuit 14 returns the processing to step S17 and continues the reverse jet. The example of FIG. 7 shows that with the flow rate F in the perfusion state detection period relatively significantly decreased as compared to the set flow rate, abnormality of the perfusion state has been detected, and as a result, the reverse jet was performed three times. In a case where the specified number of times is set to be three, when three times of reverse jet ends, the perfusion state detection circuit 14 returns the processing to step S14. In this manner, the same processing is repeated thereafter. The example of FIG. 7 shows that the three times of reverse jet in a row was performed twice.

Note that in the flow of FIG. 8, also in a case where the periodical reverse jet is set to be OFF, when abnormality of the perfusion state is determined in step S16, in steps S17, S18, the reverse jet is continuously performed only a specified number of times. The number of times of the reverse jet performed in a row may be changed for when the periodical reverse jet is set to be ON and when the periodical reverse jet is set to be OFF.

In this manner, in the present modification, by periodically performing the reverse jet, the suction conduit can be effectively prevented from being clogged. Further, when the caught calculus is not removed even though the reverse jet is periodically performed, continuous reverse jet is performed, so that the suction conduit can be surely prevented from being clogged.

### (Modification)

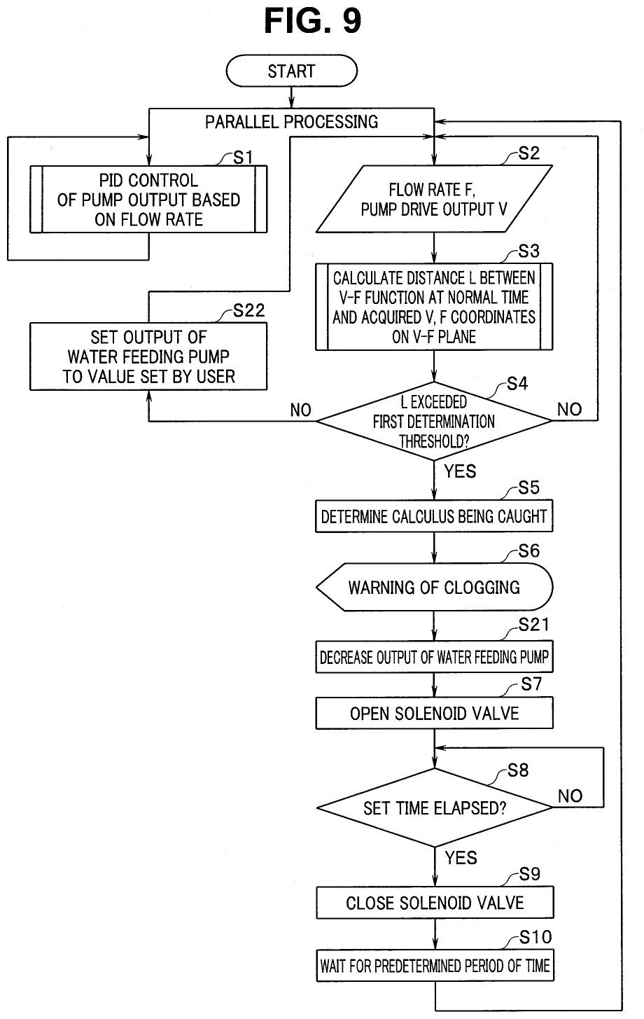

FIG. 9 is a flowchart for explaining operation of another modification. The hardware configuration of the present modification is the same as the hardware configuration of the first embodiment and the technique of detecting abnormality of the perfusion state is also the same as the technique of the first embodiment. In the present modification, when abnormality of the perfusion state is detected, the water feeding amount is reduced. In FIG. 9, for the same procedures as the procedures of FIG. 6, the same reference signs are assigned and the description will be omitted.

When as a result of calculus being caught in the suction conduit, the perfusion state detection circuit 14 detects that abnormality has occurred in the perfusion state, in steps S7 to S9, the reverse jet is performed. This could remove the calculus being caught, but when the flow rate F in the suction conduit is reduced in a period until the caught stone is completely removed, the amount of water inside the organ increases and the pressure inside the organ could increase. Thus, in the present modification, when the perfusion state detection circuit 14 detects that abnormality has occurred in the perfusion state, the amount of water fed by the liquid feeding pump 12a is reduced to suppress the increase in the pressure inside the organ.

The flow of FIG. 9 differs from the flow of FIG. 6 in that the processing of steps S21, S22 is added. Step S21 is processing of decreasing the output of the liquid feeding pump 12a when the distance L exceeds the first determination threshold and abnormality of the perfusion state is detected. When the control circuit 11 is provided with the detection result indicating the abnormality of the perfusion state from the perfusion state detection circuit 14, the control circuit 11 controls the liquid feeding pump 12a to decrease the output. This reduces the feeding amount of the liquid to be supplied to the inside of the organ, so that the pressure inside the organ is prevented from increasing.

Note that when the perfusion state detection circuit 14 determines that the perfusion state has returned to normal (NO determination of step S4), the control circuit 11, in step S22, sets the output of the liquid feeding pump 12a to the value set by the user and then returns the processing to step S2.

In this manner, according to the present modification, the pressure inside the organ can be prevented from increasing.

Note that the modification of FIG. 9 shows the example applied to the first embodiment of FIG. 6, but can also be applied to the modification of FIG. 8.

### Second Embodiment

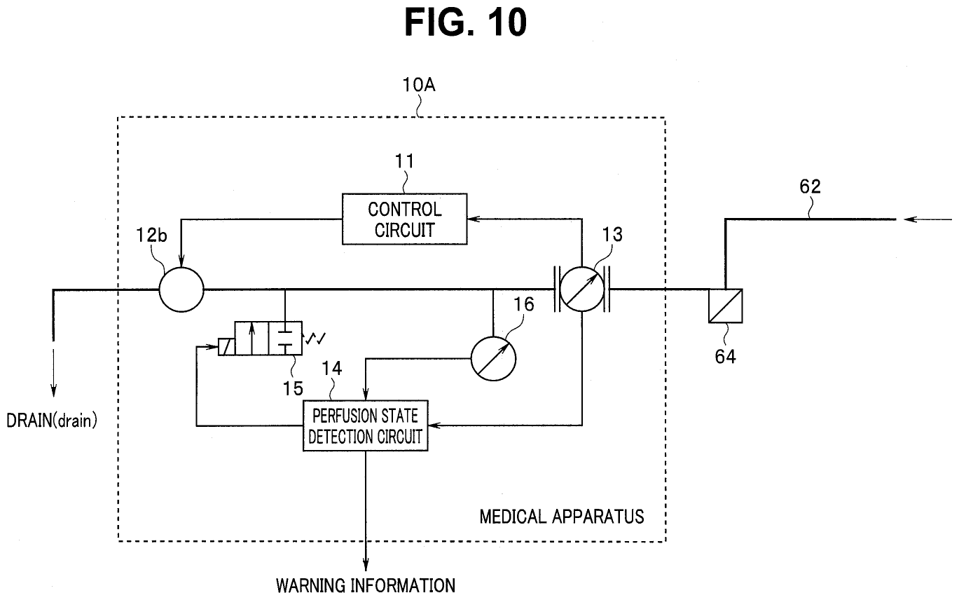

FIG. 10 is a block diagram showing a second embodiment of the present invention. In FIG. 10, for the same constituent elements as the constituent elements of FIG. 2, the same reference signs are assigned and the description will be omitted. The present embodiment detects the perfusion state based on the relation between the flow rate in the suction conduit and the suction pressure, so that the calculus being caught can be detected at an early stage.

A medical apparatus 10A of the present embodiment differs from the medical apparatus 10 of FIG. 2 in that a pressure meter 16 is added and the output from the pressure meter 16 is supplied to the perfusion state detection circuit 14, in place of the drive output V. The other components are the same as the components of the first embodiment. The present embodiment differs from the first embodiment in the technique of detecting the perfusion state. The pressure meter 16 measures the pressure inside the suction conduit and outputs the measurement result to the perfusion state detection circuit 14.

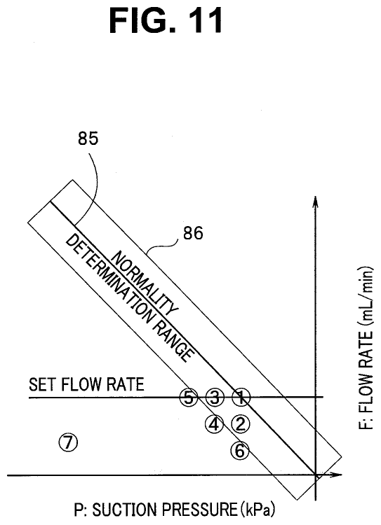

FIG. 11 is an explanatory view for explaining a technique of perfusion state detection by the perfusion state detection circuit 14 in the second embodiment. FIG. 11 shows the relation between a suction pressure P and the flow rate F on a P-F plane with the suction pressure P(kPa) by the suction pump 12b plotted on the lateral axis and with the flow rate F (mL/min) of the liquid flowing through the suction conduit plotted on the longitudinal axis. When the conduit resistance of the suction conduit is constant, the flow rate F also changes proportionally to the increase and decrease in the suction pressure P by the suction pump 12b. A straight line 85 of FIG. 11 shows a P-F characteristic curve of the initial conduit resistance at normal perfusion state.

In other words, when the P-F characteristic of the suction pressure P obtained by the output from the pressure meter 16 and the flow rate F obtained by the output from the flow meter 13 corresponds to the characteristic of the straight line 85, the conduit resistance of the suction conduit is assumed to be the initial conduit resistance at normal state. In other words, in this case, it is assumed that the conduit resistance is not increased due to calculus or the like being caught inside the suction conduit. For such detection of the perfusion state, the perfusion state detection circuit 14 obtains the relation between the suction pressure P obtained by the output from the pressure meter 16 and the flow rate F obtained by the output from the flow meter 13.

In the present embodiment, for a range within a predetermined distance (hereinafter, referred to as a second determination threshold) from the straight line 85 indicating that the conduit resistance of the suction conduit is at normal state, that is, for a normality determination range 86 of FIG. 11, it is determined that change in the conduit resistance is in the normal range. Note that the normality determination range 86 takes into consideration the fluctuation of the conduit resistance due to normal bending of the insertion portion 21 or the like. The range of the normality determination range 86, that is, the degree of the second determination threshold can be appropriately set and changed. With the second determination threshold appropriately set, it is possible to adjust the degree of catching to be determined to be abnormal.

The perfusion state detection circuit 14 determines whether the obtained characteristic value of the suction pressure P-flow rate F is included in the normality determination range 86. Note that the perfusion state detection circuit 14 may be configured to read, from a memory which is not shown, the second determination threshold for determining whether the characteristic value is included in the normality determination range 86. The user such as the operator may be able to set and change the second determination threshold by means of an input apparatus which is not shown.

In the present embodiment also, abnormality of perfusion is determined by combining the suction pressure P and the flow rate F, and abnormality of perfusion is determined without being interfered by a complicated flow and abnormality of perfusion that is merely only a partial clogging of the suction conduit by the calculus or the like can also be detected.

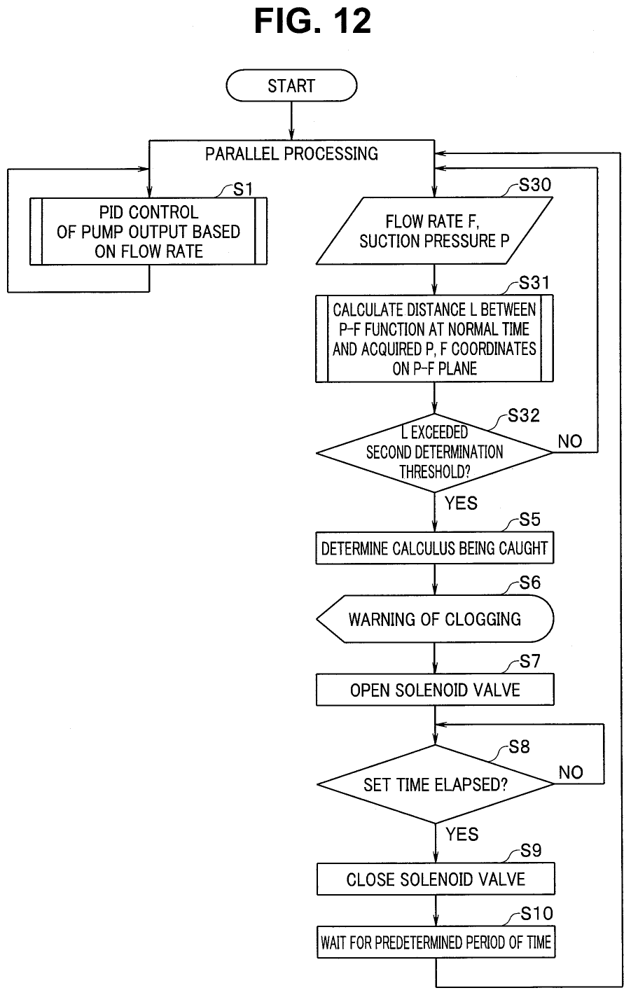

Next, the operation of the embodiment configured as such will be described with reference to FIG. 11 and FIG. 12. FIG. 12 is a flowchart for explaining perfusion control by the medical apparatus 10A. In FIG. 12, for the same procedures as the procedures of FIG. 6, the same reference signs are assigned and the description will be omitted. The flow of FIG. 12 differs from the flow of FIG. 6 in that steps S30, S31, S32 are adopted in place of steps S2, S3, S4, respectively.

Now, for example, the coordinate value on the P-F plane of the suction pressure P and the flow rate F acquired by the perfusion state detection circuit 14 is denoted by a circled FIG. 1 of FIG. 11. Note that the set flow rate is the set flow rate shown in FIG. 11. The control circuit 11 performs feedback control of the suction pressure P based on the measurement result from the flow meter 13 for maintaining the set flow rate (step S1 of FIG. 12). With this control, when the conduit resistance of the suction conduit does not change, the suction pressure P-flow rate F takes the coordinate value on the straight line 85 of FIG. 11 in accordance with the change in the set flow rate.

Here, for example, because of the normal bending operation of the insertion portion 21 or the like, the conduit resistance of the suction conduit increases from the initial conduit resistance. Thus, as shown in a circled FIG. 2 of FIG. 11, when the suction pressure P does not change, the flow rate F decreases. However, with the feedback control by the control circuit 11, the suction pressure P increases (negative pressure increases) and irrespective of the change in the conduit resistance, the flow rate F returns to the set flow rate as shown in a circled FIG. 3 of FIG. 11. When the conduit resistance increases due to the normal bending operation of the insertion portion 21 or the like, the conduit resistance occasionally returns to the original initial conduit resistance, and in this case, with the feedback control by the control circuit 11, the suction pressure P and the flow rate F return to the coordinate value of the circled FIG. 1.

However, when the increase in the initial conduit resistance results from the calculus being caught inside the suction conduit or the like, subsequently, starting from the calculus being first caught, more calculi are caught and whereby the conduit resistance further increases in some cases. If so, the flow rate F decreases as shown in a circled FIG. 4 of FIG. 11, and with the feedback control by the control circuit 11, the suction pressure P increases (circled FIG. 5) and the set flow rate is maintained.

In the present embodiment, in step S30 of FIG. 12, the flow meter 13 measures the flow rate F of the liquid flowing in the suction conduit, and the pressure meter 16 measures the suction pressure P in the suction conduit. The flow rate F and the suction pressure P are supplied to the perfusion state detection circuit 14. The perfusion state detection circuit 14 calculates, in step S31, a distance L between the coordinate of the suction pressure P-flow rate F and the straight line 85. When the relation of the suction pressure P-the flow rate F deviates from the normality determination range 86, that is, when the distance L between the coordinate of the suction pressure P-flow rate F and the straight line 85 has exceeded the second determination threshold, the perfusion state detection circuit 14 determines that abnormality has occurred in the perfusion, that is, calculus has been caught (YES determination of S32). Note that when it is determined that the distance L is within the second determination threshold (NO determination of S32), the perfusion state detection circuit 14 returns the processing from step S32 to step S2.

In the example of FIG. 11, when the coordinate value of the suction pressure P-flow rate F is on the circled FIGS. 4, 5, the perfusion state detection circuit 14 determines in step S5 that calculus has been caught in the suction conduit. Note that when the flow rate does not return to the set flow rate even with the PID control, when more calculi are caught in a short period of time, and the like, the suction pressure P-flow rate F is occasionally shifted to the coordinate position of a circled FIG. 6 from the circled FIG. 1 of FIG. 11. Even in such cases, the perfusion state detection circuit 14 can determine that calculus has been caught, in a short period of time after the occurrence of such a failure.

Note that it is also conceivable that despite the suction pressure P having reached the maximum value with the PID control by the control circuit 11, the flow rate does not return to the set flow rate and the suction pressure P-flow rate F is at the coordinate position of a circled FIG. 7 of FIG. 11. In this case also, the perfusion state detection circuit 14 can determine that calculus has been caught, before the suction conduit is completely clogged.

The processing in the perfusion state detection circuit 14 when abnormality of the perfusion state is detected, such as a case in which calculus has been caught, is the same as the processing of the first embodiment.

As described above, in the present embodiment, it is possible to detect the calculus being caught at an early stage by detecting the state of the perfusion based on the relation between the flow rate in the suction conduit for discharging calculus to the outside of a body and the suction pressure. The other advantageous effects are the same as the advantageous effects of the first embodiment.

Note that the modifications of FIG. 7, FIG. 8, and FIG. 9 may be applied to the present embodiment.

Further, in the second embodiment, the example of detecting the perfusion state based on the relation between the suction pressure P and the flow rate F has been shown, but since the suction pump is subjected to the PID control, when the set value of the flow rate is constant, it is also possible to detect abnormality of the perfusion state by simply comparing the suction pressure P with a predetermined threshold and based on whether the suction pressure P has exceeded the predetermined threshold.

### (Modification)

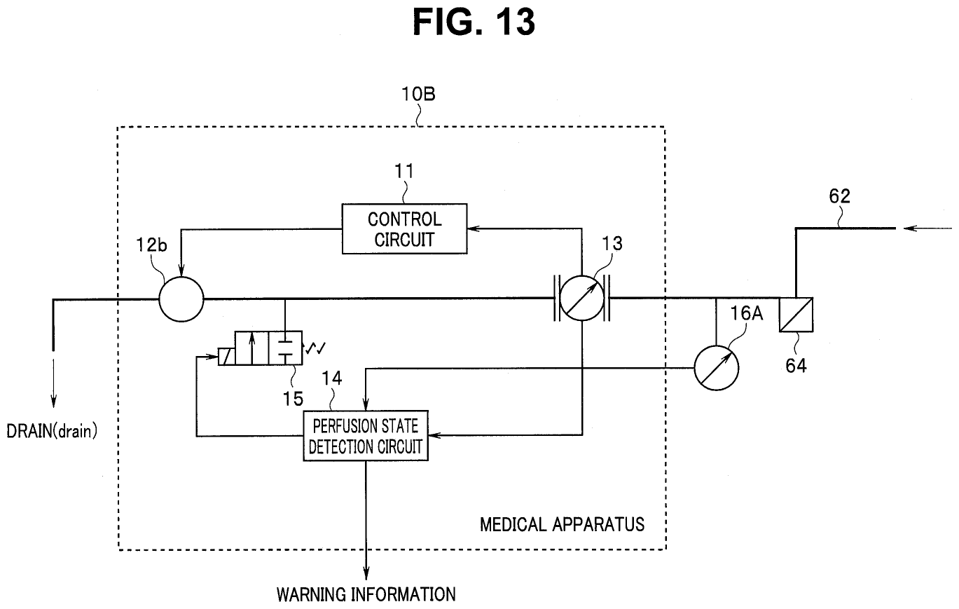

FIG. 13 is a block diagram showing another modification. In FIG. 13, for the same constituent elements as the constituent elements of FIG. 11, the same reference signs are assigned and the description will be omitted.

The example of FIG. 13 adopts a medical apparatus 10B, which omits the pressure meter 16 of the medical apparatus 10A, and detects the suction pressure in the suction conduit using a pressure meter 16A provided on the outside of the medical apparatus 10B.

The other configurations, functions, and advantageous effects are the same as the configurations, functions, and advantageous effects of the embodiment of FIG. 10.

### (Modification)

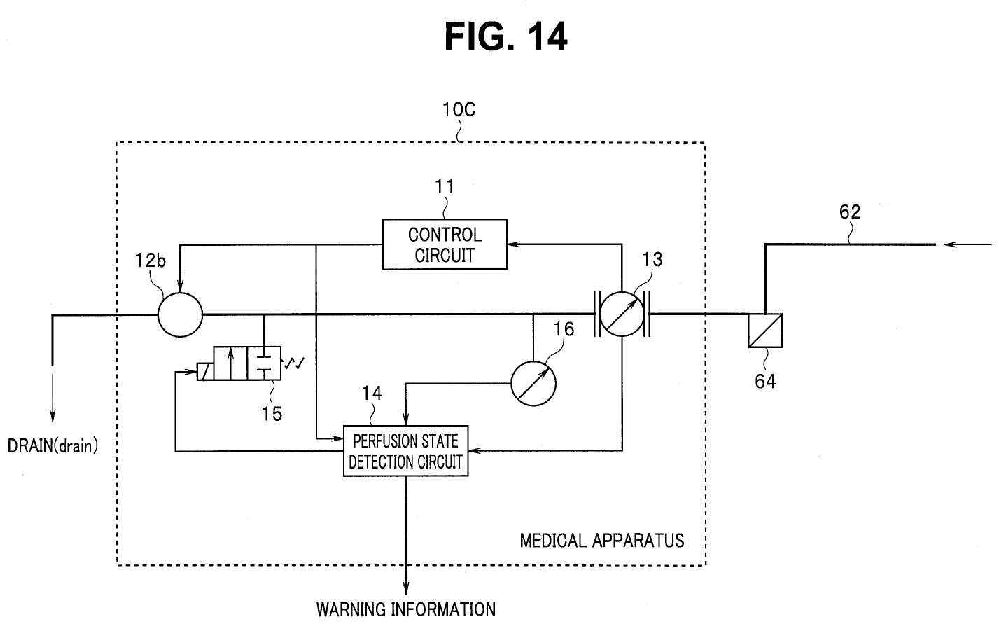

FIG. 14 is a block diagram showing another modification. In FIG. 14, for the same constituent elements as the constituent elements of FIG. 2 and FIG. 10, the same reference signs are assigned and the description will be omitted. The present modification performs both the abnormality detection of the perfusion state based on the relation of the drive output V-the flow rate F and the abnormality detection of the perfusion state based on the relation of the suction pressure P-the flow rate F, by combining the first and second embodiments.

A medical apparatus 10C of FIG. 14 differs from the medical apparatuses 10, 10A of FIG. 2 and FIG. 10 in that the flow rate F from the flow meter 13, the drive output V from the control circuit 11, and the suction pressure P from the pressure meter 16 are provided to the perfusion state detection circuit 14. The perfusion state detection circuit 14 performs the abnormality detection of the perfusion state using the relation of the drive output V-the flow rate F and the abnormality detection of the perfusion state using the relation of the suction pressure P-the flow rate F.

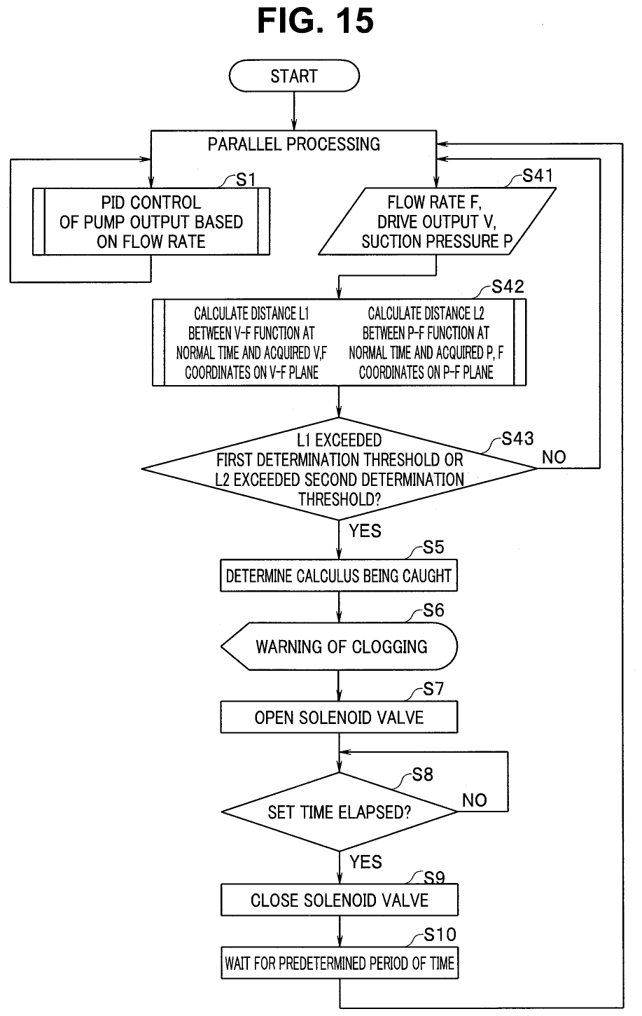

Next, the operation of the embodiment configured as such will be described with reference to FIG. 15. FIG. 15 is a flowchart for explaining operation of the modification of FIG. 14. In FIG. 15, for the same procedures as the procedures of FIG. 6 and FIG. 12, the same reference signs are assigned and the description will be omitted.

The flow meter 13 measures the flow rate F of the liquid flowing through the suction conduit and outputs the measurement result to the control circuit 11 and the perfusion state detection circuit 14. The control circuit 11 provides the drive output V set for the suction pump 12b to the perfusion state detection circuit 14. Further, the pressure meter 16 measures the suction pressure P in the suction conduit and provides the suction pressure P to the perfusion state detection circuit 14. In this manner, the flow rate F, the drive output V, and the suction pressure P are inputted to the perfusion state detection circuit 14 (step S41 of FIG. 15).

The perfusion state detection circuit 14 calculates, in step S42, a distance L1 between the V-F function at normal time on the V-F plane indicated by the straight line 81 of FIG. 5 and the acquired coordinate value of the drive output V and the flow rate F, and calculates a distance L2 between a P-F function at normal time on the P-F plane indicated by the straight line 85 of FIG. 11 and the acquired coordinate value of the suction pressure P and the flow rate F.

The perfusion state detection circuit 14 determines whether the distance L1 has exceeded the first determination threshold and determines whether the distance L2 has exceeded the second determination threshold. When the distance L1 has exceeded the first determination threshold and/or the distance L2 has exceeded the second determination threshold, the perfusion state detection circuit 14 determines that abnormality has occurred in the perfusion state (YES determination of step S43), and shifts the processing to step S5. Further, when the distance L1 is within the first determination threshold and the distance L2 is within the second determination threshold, the perfusion state detection circuit 14 determines that no abnormality has occurred in the perfusion state (NO determination of step S43), and returns the processing to step S41.

The other functions are the same in the flows of FIG. 6 and FIG. 12.

In this manner, the present modification determines the abnormality of the perfusion state using the detection results of both the abnormality detection of the perfusion state based on the relation of the drive output V-the flow rate F and the abnormality detection of the perfusion state based on the relation of the suction pressure P-the flow rate F, and even though the calculus being caught is minor, the catching can be detected at an early stage.

### Third Embodiment

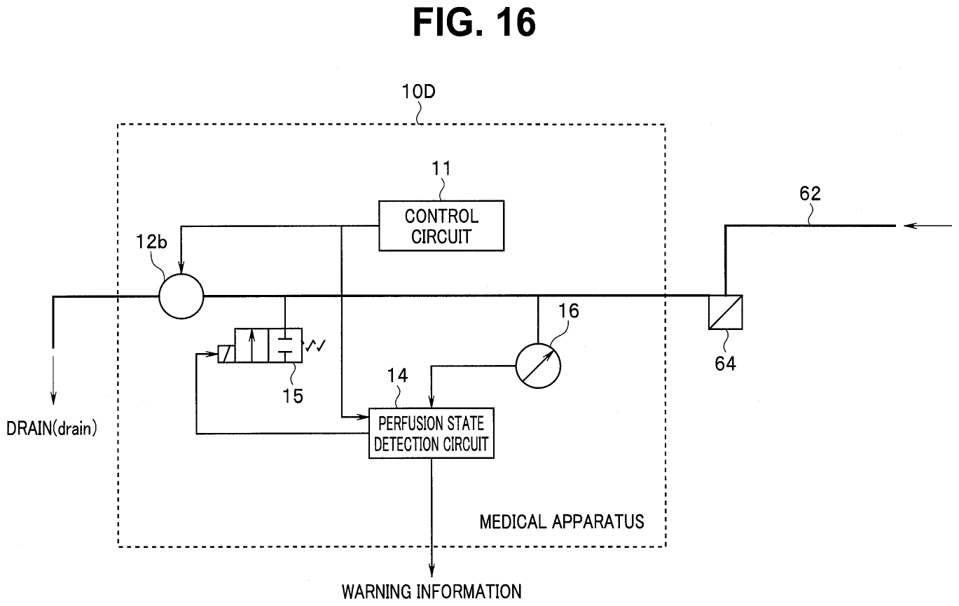

FIG. 16 is a block diagram showing a third embodiment of the present invention. In FIG. 16, for the same constituent elements as the constituent elements of FIG. 14, the same reference signs are assigned and the description will be omitted. The present embodiment detects the perfusion state based on the relation between the suction pressure in the suction conduit for discharging calculus to the outside of a body and the drive output to the suction pump 12b, so that the calculus being caught can be detected at an early stage.

A medical apparatus 10D of the present embodiment differs from the medical apparatus 10A of FIG. 10 in that the flow meter 13 is omitted and the control circuit 11 does not perform the PID control. The other components are the same as the components of the modification of FIG. 14. The present embodiment differs from each of the aforementioned embodiments in the technique of detecting the perfusion state.

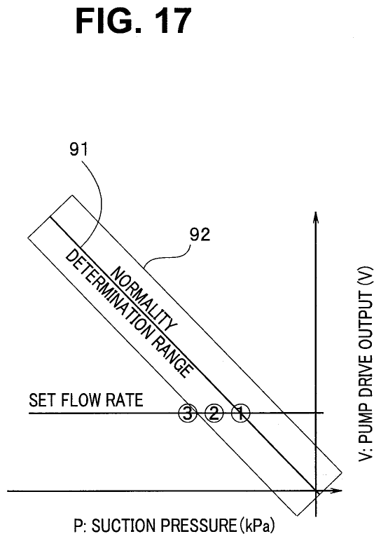

FIG. 17 is an explanatory view for explaining a technique of perfusion state detection by the perfusion state detection circuit 14 in the third embodiment. FIG. 17 shows the relation between the suction pressure P and the drive output V on a P-V plane with the suction pressure P(kPa) by the suction pump 12b plotted on the lateral axis and with the drive output V(V) to the suction pump 12b plotted on the longitudinal axis. In the present embodiment, the control circuit 11 does not perform the PID control of feedback of the flow rate of the liquid flowing through the suction conduit and the drive output V set by a user is outputted to the suction pump 12b.

When the conduit resistance of the suction conduit is constant, the suction pressure P by the suction pump 12b also changes proportionally to the increase and decrease in the drive output V to the suction pump 12b. A straight line 91 of FIG. 17 shows a P-V characteristic curve of the initial conduit resistance at normal perfusion state.

In other words, when the P-V characteristic of the suction pressure P obtained by the output from the pressure meter 16 and the drive output V based on the output from the control circuit 11 corresponds to the characteristic of the straight line 91, the conduit resistance of the suction conduit is assumed to be the initial conduit resistance at normal state. In other words, in this case, it is assumed that the conduit resistance is not increased due to calculus or the like being caught inside the suction conduit. For such detection of the perfusion state, the perfusion state detection circuit 14 obtains the relation between the suction pressure P obtained by the output from the pressure meter 16 and the drive output V obtained by the output from the control circuit 11.

In the present embodiment, for a range within a predetermined distance (hereinafter, referred to as a third determination threshold) from the straight line 91 indicating that the conduit resistance of the suction conduit is at normal state, that is, a normality determination range 92 of FIG. 17, it is determined that change in the conduit resistance is in the normal range. Note that the normality determination range 92 takes into consideration the fluctuation of the conduit resistance due to normal bending of the insertion portion 21 or the like. The range of the normality determination range 92, that is, the degree of the third determination threshold can be appropriately set and changed. With the third determination threshold appropriately set, it is possible to adjust the degree of catching to be determined to be abnormal.

The perfusion state detection circuit 14 determines whether the obtained characteristic value of the suction pressure P-drive output V is included in the normality determination range 92. Note that the perfusion state detection circuit 14 may be configured to read, from a memory which is not shown, the third determination threshold for determining whether the characteristic value is included in the normality determination range 92. The user such as the operator may be able to set and change the third determination threshold by means of an input apparatus which is not shown.

In the present embodiment also, abnormality of perfusion is determined by combining the suction pressure P and the drive output V, and abnormality of perfusion is determined without being interfered by a complicated flow and abnormality of perfusion that is merely only a partial clogging of the suction conduit by the calculus or the like can also be detected.

In the embodiment configured as such, abnormality of the perfusion state is detected by the same flow as the flow of each of the aforementioned embodiments.

Now, for example, the coordinate value on the P-V plane of the suction pressure P and the drive output V acquired by the perfusion state detection circuit 14 is denoted by a circled FIG. 1 of FIG. 17. Note that the set flow rate is the set flow rate shown in FIG. 17. The control circuit 11 does not perform the PID control and the drive output V to the suction pump 12b is a value set by a user. In the example of FIG. 17, for example, in the initial conduit resistance, the drive output V to obtain the set flow rate is set.

Here, for example, because of the normal bending operation of the insertion portion 21 or the like, the conduit resistance of the suction conduit increases from the initial conduit resistance. Thus, as shown in a circled FIG. 2 of FIG. 17, the suction pressure P increases (negative pressure increases). Note that when the conduit resistance increases due to the normal bending operation of the insertion portion 21 or the like, the conduit resistance occasionally returns to the original initial conduit resistance, and in this case, the suction pressure P and the drive output V return to the coordinate value of the circled FIG. 1.

However, when the increase in the initial conduit resistance results from the calculus being caught inside the suction conduit or the like, subsequently, starting from the calculus being first caught, more calculi are caught and whereby the conduit resistance further increases in some cases. If so, the suction pressure P further increases as shown in a circled FIG. 3 of FIG. 17.

In the present embodiment, the perfusion state detection circuit 14 calculates a distance L between the coordinate of the suction pressure P-drive output V and the straight line 91. When the relation of the suction pressure P-the drive output V deviates from the normality determination range 92, that is, when the distance L between the coordinate of the suction pressure P-drive output V and the straight line 91 has exceeded the third determination threshold, the perfusion state detection circuit 14 determines that abnormality has occurred in the perfusion, that is, calculus has been caught. Note that when it is determined that the distance L is within the third determination threshold, the perfusion state detection circuit 14 determines that the perfusion state is normal.

The processing in the perfusion state detection circuit 14 when abnormality of the perfusion state is detected, such as a case in which calculus has been caught, is the same as the processing of each of the aforementioned embodiments.

In this manner, in the present embodiment, the perfusion state is detected based on the relation between the suction pressure in the suction conduit for discharging calculus to the outside of a body and the drive output, so that the calculus being caught can be detected at an early stage. The other advantageous effects are the same as the advantageous effects of each of the aforementioned embodiments.

Note that in the third embodiment, the example of detecting the perfusion state based on the relation between the suction pressure P and the drive output V has been shown, but when the set value of the drive output V is constant, it is also possible to detect abnormality of the perfusion state by simply comparing the suction pressure P with a predetermined threshold and based on whether the suction pressure P has exceeded the predetermined threshold. Further, in the case of performing the PID control of the suction pump also, it is possible to detect the perfusion state based on the relation between the suction pressure P and the drive output V.

The present invention is not limited to each of the exact aforementioned embodiments, but can be embodied by modifying the constituent elements within the scope without departing from the gist of the present invention at the implementing stage. Further, various inventions can be formed by appropriately combining a plurality of constituent elements disclosed in each of the aforementioned embodiments. For example, some constituent elements of all the constituent elements shown in the embodiments may be deleted. Further, the constituent elements of different embodiments may be combined as appropriate.

For example, in each of the aforementioned embodiments, the examples of detecting abnormality of the perfusion state in the suction conduit at an early stage have been described, but the present invention is also applicable to early detection of abnormality of the perfusion state in the liquid feeding conduit.

What is claimed is:

1. A perfusion state detection method, comprising:
driving a liquid feeding pump configured to supply a liquid via a liquid feeding conduit inserted into an inside of a living body;
driving a suction pump configured to discharge the liquid via a suction conduit inserted into the inside of the living body;
obtaining a distance, on a plane indicating a relation between any two values of a drive output to the suction pump, a flow rate of the liquid flowing through the conduit, and a pressure in the conduit, between a coordinate on the plane of the two values in a perfusion state at normal time of the conduit and a coordinate on the plane of the two values actually measured;
detecting abnormality of the perfusion state of the conduit based on whether the distance exceeds a threshold; and
controlling a flow of the liquid in the conduit based on a detection result of the perfusion state.

2. The perfusion state detection method according to claim 1, comprising generating reverse jet in the suction conduit to control the flow of the liquid in the suction conduit.

3. The perfusion state detection method according to claim 2, comprising reducing an amount of the liquid caused to flow to the liquid feeding conduit to control the flow of the liquid in the liquid feeding conduit.

4. The perfusion state detection method according to claim 2, comprising controlling a valve to open and close to generate a water hammering action in the suction conduit so as to generate the reverse jet in the suction conduit.

5. The perfusion state detection method according to claim 1, comprising performing feedback control to make the flow rate constant by controlling the drive output of the suction pump based on the flow rate.

6. The perfusion state detection method according to claim 4, comprising bringing the valve into an open state when a detection result indicating the perfusion state being abnormal is obtained in a closed state of the valve.

7. The perfusion state detection method according to claim 4, comprising bringing the valve into a closed state when a detection result indicating the perfusion state being abnormal is obtained in an open state of the valve.

8. The perfusion state detection method according to claim 1, comprising measuring the flow rate of the liquid flowing through the suction conduit, wherein detection of the perfusion state includes procedures of:
obtaining a distance, on a drive output-flow rate plane indicating a relation between the drive output and the flow rate, between a coordinate on the drive output-flow rate plane of the drive output and the flow rate in the perfusion state at normal time of the conduit and a coordinate on the drive output-flow rate plane of the drive output and the flow rate that are actual drive output and flow rate; and
determining abnormality of the perfusion state based on whether the distance exceeds a first threshold.

9. The perfusion state detection method according to claim 1, comprising measuring the flow rate of the liquid flowing through the suction conduit, wherein
detection of the perfusion state includes procedures of:
obtaining a distance, on a pressure-flow rate plane indicating a relation between the pressure and the flow rate, between a coordinate on the pressure-flow rate plane of the pressure and the flow rate in the perfusion state at normal time of the conduit and a coordinate on the pressure-flow rate plane of the pressure and the flow rate that are actual pressure and flow rate; and
determining abnormality of the perfusion state based on whether the distance exceeds a second threshold.

10. The perfusion state detection method according to claim 1, comprising measuring the pressure of the liquid flowing through the suction conduit, wherein
detection of the perfusion state includes procedures of:
obtaining a distance, on a pressure-drive output plane indicating a relation between the pressure and the drive output, between a coordinate on the pressure-drive output plane of the pressure and the drive output in the perfusion state at normal time of the conduit and a coordinate on the pressure-drive output plane of the pressure and the drive output that are actual pressure and drive output; and
determining abnormality of the perfusion state based on whether the distance exceeds a third threshold.

11. A perfusion state detection apparatus comprising:
a liquid feeding conduit inserted into an inside of a living body and configured to feed a liquid to the inside of the living body;
a liquid feeding pump configured to supply the liquid to the liquid feeding conduit;
a suction conduit inserted into the inside of the living body and configured to suck the liquid from the inside of the living body;
a suction pump configured to discharge the liquid from the inside of the living body via the suction conduit;
a valve configured to generate reverse jet in the suction conduit by a water hammering action; and
a processor
wherein
the processor:
obtains a distance, on a plane indicating a relation between any two values of a drive output to the suction pump, a flow rate of the liquid flowing through the suction conduit, and a suction pressure in the suction conduit, between a coordinate on the plane of the two values in a perfusion state at normal time of the conduit and a coordinate on the plane of the two values actually measured;
detects abnormality of the perfusion state of the suction conduit based on whether the distance exceeds a first threshold; and
controls opening and closing of the valve based on a detection result of the perfusion state to generate the reverse jet in the suction conduit.

12. The perfusion state detection apparatus according to claim 11,
wherein
the suction conduit comprises a bypass portion, and
the valve is connected to a terminal end of the bypass portion and opens the suction conduit to generate the reverse jet.

13. The perfusion state detection apparatus according to claim 11, comprising a flow meter provided in the suction conduit, the flow meter configured to measure the flow rate of the liquid flowing through the suction conduit,
wherein
the processor:
obtains a distance, on a drive output-flow rate plane indicating a relation between the drive output and the flow rate, between a coordinate on the drive output-flow rate plane of the drive output and the flow rate in the perfusion state at normal time of the conduit and a coordinate on the drive output-flow rate plane of the drive output and the flow rate that are actual drive output and flow rate; and
determines abnormality of the perfusion state based on whether the distance exceeds the first threshold.

14. The perfusion state detection apparatus according to claim 11, comprising a flow meter provided in the suction conduit, the flow meter configured to measure the flow rate of the liquid flowing through the suction conduit,
wherein
the processor:
obtains a distance, on a suction pressure-flow rate plane indicating a relation between the suction pressure and the flow rate, between a coordinate on the suction pressure-flow rate plane of the suction pressure and the flow rate in the perfusion state at normal time of the conduit and a coordinate on the suction pressure-flow rate plane of the suction pressure and the flow rate that are actual suction pressure and flow rate; and
determines abnormality of the perfusion state based on whether the distance exceeds a second threshold.

15. The perfusion state detection apparatus according to claim 11, comprising a pressure meter provided in the suction conduit, the pressure meter configured to measure the suction pressure of the liquid flowing through the suction conduit,
wherein
the processor:
obtains a distance, on a suction pressure-drive output plane indicating a relation between the suction pressure and the drive output, between a coordinate on the suction pressure-drive output plane of the suction pressure and the drive output in the perfusion state at normal time of the conduit and a coordinate on the suction pressure-drive output plane of the suction pressure and the drive output that are actual suction pressure and drive output; and
determines abnormality of the perfusion state based on whether the distance exceeds a third threshold.

16. The perfusion state detection apparatus according to claim 11, wherein the processor reduces the drive output of the liquid feeding pump that causes the liquid to flow to the liquid feeding conduit, when it is determined that abnormality occurs in the perfusion state.

17. An endoscope system comprising:
an endoscope;
a liquid feeding pump configured to cause a liquid to flow to a liquid feeding conduit of the endoscope;
a suction pump configured to cause the liquid to flow via a suction conduit of the endoscope;
a flow meter provided in the suction conduit and configured to measure a flow rate of the liquid flowing through the suction conduit;
a valve configured to generate reverse jet in the suction conduit by a water hammering action; and
a processor
wherein
the processor
obtains a distance, on a plane indicating a relation between any two values of a drive output to the suction pump, the flow rate of the liquid flowing through the suction conduit, and a suction pressure in the suction conduit, between a coordinate on the plane of the two values in a perfusion state at normal time of the conduit and a coordinate on the plane of the two values actually measured;
detects abnormality of the perfusion state of the suction conduit based on whether the distance exceeds a first threshold; and
controls opening and closing of the valve based on a detection result of the perfusion state to generate the reverse jet in the suction conduit.
